(19) United States
(12) Patent Application Publication
(10) Pub. No.: US 2006/0137981 A1
(43) Pub. Date: Jun. 29, 2006

(54) Nanoelectrode device for chemical analysis

(76) Inventors: Kristinn Johnsen, Seltjarnanes (IS); Sveinn Olafsson, Kopavogur (IS)

Correspondence Address: HARNESS, DICKEY & PIERCE, P.L.C., P.O. BOX 8910, RESTON, VA, 20195, US

(21) Appl. No.: 10/531,416

(22) PCT Filed: Oct. 17, 2003
(86) PCT No.: PCT/IS03/00031

§ 371 Date: Apr. 15, 2005

(30) Foreign Application Priority Data
Oct. 18, 2002 (IS) 6584

(51) Int. Cl. C07K 1/26; B03C 5/02; G01N 27/447

(52) U.S. Cl. 204/450; 204/600; 204/547; 204/643

(57) ABSTRACT

A device is provided with tunable affinity for molecules such as in particular macromlecues including proteins and peptides. The device comprises a substrate with a surface; a plurality of locally substantially parallel electrodes along said surface, wherein adjacent electrodes are separated by a distance in the range of about 0.1 nm to about 1 μm, such as about 0.3 nm to about 10 nm, where each of said electrodes is connected to a tunable EMF source, such that a specific electrostatic environment perpendicular to said electrodes is created, extending along the electrodes, and providing a continuous binding area for molecules in contact with the binding area. By tuning the independently tunable EMF sources a specific affinity or repulsion is obtainable for molecules with specific electrostatic properties. Also provided are methods for separating and isolating molecules with a device such as is disclosed herein.

### BACKGROUND OF THE INVENTION

Detection, identification and quantification of proteins and other complex macromolecules is critical to many fields within the chemical, medical and pharmaceutical industries and sciences, as well as food manufacturing. For example, diagnostic assays may include the quantification of specific protein(s), DNA or other macromolecules in a biological sample. Other uses include the identification and quantification of specific pathogens in biological or environmental samples, as well as in vitro determination of macromolecular composition in samples of various nature, both liquid and gaseous. The latter application is particularly useful in the food industry, but is increasingly being utilized in other fields, including medicine. Thus, it is well known that certain animals are capable of sensing minute changes in odour, leading to a specific physiological response (e.g., fear conditioning). Furthermore, it is believed that odour changes may accompany certain physiological changes, such as malignant cell growth. These changes in odour are believed to be due to increased production of specific organic and/or biological molecules, resulting in an increased interest in methods of their detection, both in vivo and in vitro.

Qualitative and quantitative assays of individual macromolecules are currently complex and generally require sophisticated and bulky equipment Currently common techniques that are used for detection of macromolecules such as proteins and nucleic acids include spectrophotometric assays, immunoassays, enzymatic assays, liquid chromatography (LC), mass spectroscopy (MS), electrophoresis, micro array techniques, and various combinations of these methods.

Other recent techniques for macromolecule analysis include high density DNA chips such as built by Affymetrix as originally described in PCT International Publication No. WO 90/15070.

U.S. Pat. No. 5,624,537, entitled “BIOSENSOR AND INTERFACE MEMBRANE”, describes a protein-receiving matrix and a single electrode.

U.S. Pat. No. 5,395,587, entitled “SURFACE PLASMON RESONANCE DETECTOR HAVING COLLECTOR FOR ELUTED LIGATE”, describes a system to measure immobilized ligands using a plasmon resonance detector.

U.S. Pat. No. 5,328,847 entitled “THIN MEMBRANE SENSOR WITH BIOCHEMICAL SWITCH”, describes a biosensor with a specific recognition for biomolecules.

U.S. Pat. No. 4,777,019 entitled “BIOSENSOR”, describes a biosensor for biological monomers.

U.S. Pat. No. 5,532,128, entitled “MULTI-SITE DETECTION APPARATUS”, describes test wells combined with electrodes to detect given biological molecules.

U.S. Pat. No. 5,384,028 entitled “BIOSENSOR WITH A DATA MEMORY”. describes a membrane biosensor with a memory module.

U.S. Pat. No. 4,981,572 entitled “ELECTRODE UNIT AND PACKAGE FOR A BLOOD ANALYZER”, describes an electrode and apparatus to analyze blood.

U.S. Pat. No. 4,908,112 entitled “SILICON SEMICONDUCTOR WAFER FOR ANALYZING MICRONIC BIOLOGICAL SAMPLES”, describes a micro capillary separation device with detector capabilities.

U.S. Pat No. 5,409,583 entitled “METHOD FOR MEASURING CONCENTRATIONS OF SUBSTRATES IN A SAMPLE LIQUID BY USING A BIOSENSOR”, describes a two step biosensor.

U.S. Pat. No. 6,123,819 entitled NANOELECTRODE ARRAYS, describes an array of nanoelectrodes extending from a principal surface and having a varied spatial distribution, height, width and electrochemical composition to provide specific electronic receptors.

In general, the above described technologies are used for the detection of a single type or a few different types of molecules. The techniques have different advantages and disadvantages, and there still exists a need for general broad-based methods for the detection of macromolecules that do not require specific substances or substrates for a specific type of macromolecule to be assessed, and that allow for high-throughput analysis of samples with low-cost non-spacious equipment.

### SUMMARY OF THE INVENTION

The invention is based on the feature of most molecules that they contain an electrostatic surface that may be approximated by a set of distributed dipoles. These dipoles are characterized by the charge distribution of the molecule in question.

The main aspect of the invention is a device constructed using thin film nanotechniques. The device comprises a surface of tunable nano-electrodes which provide a molecule-specific sensor, the affinity of which can be readily altered to bind different molecules. In a preferred embodiment, the sensor surface is homogeneous in one dimension and can specifically bind or repel many identical molecules simultaneously. The surface has a plurality of locally substantially parallel electrodes along the binding surface, each of said electrodes being connected to a tunable EMF source, such that a specific electrostatic profile perpendicular to said electrodes can be created, extending along the electrodes, providing a continuous binding surface for molecules in contact with the binding area. The electromagnetic field may be time-dependent, so as to optimize binding of specific molecules to the binding surface.

The conductance between the electrodes is proportional to the number of molecules bound and is therefore a quantitative measure of the concentration of the specific macromolecule in the sample being analyzed. If the macromolecule being measured has a low or vanishing conductance the sample may be irradiated with light in such a way that a photocurrent is produced between the detection electrodes. The photocurrent is proportional to the number of bound molecules and is thereby a quantitative measure of the concentration in the sample.

In principle, any given electrode of the device of the invention can be configured as a detection electrode. The nature of any given embodiment will determine which configuration is appropriate.

The device can be configured for various applications, e.g. identification, isolation, quantitative determination and chromatography of molecules, as well as catalysis of chemical reactions. The device can in principle be adapted to any molecule which has characteristic multipole moment, but the device is preferably used for macromolecules, which are well known to contain such molecule-specific multipole moments. In this context, a macromolecule is considered as a molecule, organic or inorganic, comprising in excess of about 100 atoms. In the description of preferred embodiments of the invention that follows, specific reference to macromolecules will be made. It should however be emphasized that the invention may be adapted for the analysis of virtually any molecular species, as will be appreciated from the description that follows. The device is particularly useful for polypeptides and proteins that depending on their amino acid composition and three-dimensional structure may have a quite specific spatial electrostatic configuration. The device can also be configured for the analysis of any liquid or gaseous sample. Furthermore, the device is not limited by the composition of the sample; any sample containing a heterogeneous population of molecules may be analyzed.

It is contemplated that the device may also be configured for reversed operation in which the ATP and ADP energy cycle is used to extract current with help of the electrodes.

The invention provides in a first aspect a device with tunable affinity for molecules, comprising: a substrate with a surface; a plurality of locally substantially parallel electrodes along said surface, each of said electrodes being connected to a tunable EMF source, such that a specific electrostatic environment perpendicular to said electrodes is created, extending along the electrodes, and providing a continuous binding area for molecules in contact with the binding area; by tuning the independently tunable EMF sources a specific affinity or repulsion is obtainable for molecules with specific electrostatic properties.

In one embodiment, adjacent electrodes are separated by a distance in the range of about 0.1 nm to about 10 nm, such as in the range of about 0.2 nm to about 8 nm, and preferably in the range of about 0.3 nm to about 5 nm, including the range of about 0.5 to about 3 nm, such as e.g. about 0.05 nm or about 1 nm.

The electrodes may be each connected independently to a tunable EMF source, or one or more of the electrodes can be connected jointly to the same EMF source.

In a useful embodiment the surface is formed perpendicular along the growth direction of alternately grown layers of insulating and conducting material, such as described in the Examples herein.

In one embodiment said surface is formed by an array of canisters penetrating at least one layer of alternating insulating and conducting material, in which the inner surface of said canisters forms said surface. Such canisters are generally circular in shape but may in principle have any given shape, e.g. oval, rectangular, etc.

In preferred embodiments the device has a tunable affinity for macromolecules, in particular a macromolecule selected from polypeptides, proteins, and nucleic acids including DNA and RNA. However, the device may also be configured to provide affinity for other molecules, including carbohydrates, lipids, as well as other organic molecules.

In a further aspect, the invention sets forth a method of isolating molecules based on their electrostatic properties. The method comprises providing a substrate having a plurality of locally substantially parallel electrodes, each electrode being independently connected to a tunable EMF source; tuning said electrodes to individually selected potentials, thereby creating a specific electrostatic profile across the electrodes to generate a molecule-specific surface; contacting a medium comprising molecules with the substrate to allow binding of specific molecules having an affinity to said surface; separating the medium from the substrate so as to separate the molecules bound to the substrate from non-binding molecules in the medium; isolating said specific molecules by adjusting said EMF source to release by repulsion or by other means said molecules into a receiving medium brought in contact with the substrate.

In one embodiment the method further comprises the step of repeatedly releasing specifically bound molecules from said substrate into a receiving medium and subsequently bringing said substrate in contact with the medium containing molecules, so as to effectively remove specific molecules from the medium.

The method comprises in a useful embodiment the step of detecting said binding molecules by observing the change in current through one or more of said electrodes.

Additionally, quantitative assessment for said binding molecules can be obtained, by comparing the current necessary to maintain the electrostatic configuration of the surface to the corresponding current obtained using samples with known concentration of molecules identical to said binding molecules.

One particular embodiment comprises the step of detecting said binding molecules by irradiating the molecule with light and measuring the conductivity of said molecules. For this purpose any convenient light source emitting a light with a wavelength affecting the conductance of the molecule bound to the surface may be applied. Such method may suitably comprise the step of measuring the conductivity of said binding molecules as a function of frequency of the light (using a broadband or tunable source of light), thus generating a photo-conductance curve of said molecule.

Further, quantitative determination of said binding molecules may be obtained by mapping photo-conductance curves for macromolecular species present in the sample to photo-conductance curves of samples of known concentration containing said macromolecular species.

One embodiment comprises the step of detecting said binding molecules by irradiating the molecule with pulsed light and measuring the hopping conductance of said molecules. In such embodiment quantitative determination may be obtained by comparing the hopping conductance for the bound molecules to the hopping conductance of said molecules in a sample with known concentration of said molecules.

In another aspect of the invention, a method of catalyzing chemical reactions between molecules is provided, comprising providing a substrate having a plurality of locally substantially parallel electrodes, each electrode independently connected to a tunable EMF source; tuning said electrodes to individually selected potentials, thereby creating a specific electrostatic profile across the electrodes to generate a molecule-specific surface; contacting a medium comprising molecules with the substrate to allow binding of molecules specific for the chemical reaction and having an affinity to said surface; release of said molecules by adjustment of said EMF source into close spacial proximity of said surface; whereby a chemical reaction between said molecules is accelerated by the increase in local concentration of said molecules.

The EMF in substantially parallel electrodes may be suitably adjusted in an adjacent fashion, so as to generate adjacent specific binding sites for molecules specific for the chemical reaction.

In yet a further aspect, a method of separating molecules is provided, comprising: providing a substrate having a plurality of locally substantially parallel electrodes, each electrode being independently connected to a tunable EMF source; tuning said electrodes so as to generate a non-specific electrostatic profile across the electrodes, thus generating a non-specific macromolecular surface; placement of a micro-channel along said surface; allowing a medium comprising molecules to pass through the micro-channel, thereby allowing separation of molecules according to their dipole moment.

In one embodiment of the method, the tunable EMF is applied in a cyclic manner so as to generate a time-dependent non-specific macromolecular surface, thus allowing reversible non-specific binding to the surface

Detection of said molecules can be achieved by applying a detection technique along said micro-channel distal to said surface, said technique may be suitably selected from the group consisting of UV spectrophotometry, circular dichroism, fluorescence spectrophotometry, mass spectrometry, chemical potential, radioactivity.

The methods of the invention are particularly useful for macromolecules including polypeptides, proteins and nucleic acids including DNA, and RNA, as well as for other organic or inorganic molecules including those mentioned above.

Another aspect of the invention provides a method of producing a device with tunable affinity for molecules, the method comprising growing a superlattice structure of alternate layers of electrically insulating and conducting materials; cleaving or polishing the superlattice structure to obtain a surface essentially perpendicular to the growth direction of the superlattice structure; connecting two or more of the conducting layers individually to tunable EMF sources, to obtain substantially parallel electrodes extending along said surface, which electrodes are individually or collectively tunable to a desired potential.

The insulating layers generally have a thickness in the range of about 0.10 nm-10 μm, such as in the range of about 0.10 nm-1 μm, including about 1-100 nm, but may in particular embodiments may be even thicker; the conducting layers will similarly typically have a thickness in the range of about 0.10 nm-10 μm, such as of about 0.50 nm-1 μm, including the range of about 1-100 nm, such as e.g. in the range of about 1-50 nm.

In a useful embodiment the method further comprises the step of forming an array of canisters such as those described above penetrating at least one of said alternate layers of electrically insulating and conducting material.

### BRIEF DESCRIPTION OF THE DRAWINGS

FIG. 1 represents a surface comprised of alternating insulating (1) and conducting (2) layers. The electrochemical potential of the elecrodes (4) is adjusted by applying a voltage via nanowires (3) to the electrodes. Macromolecules (5) with a specific multipole moment are attracted by electromagnetic forces (6) to the specific dipole generated by the electrodes.

FIG. 2 illustrates an embodiment of the device, wherein N substantially parallel electrodes (4), comprised of alternating layers of conducting (1) and insulating (2) material, generate dipoles which may be adjusted so as to optimize binding of a specific macromolecule (5).

FIG. 3 illustrates how macromolecule-specific binding sites (5) are generated perpendicular to the growth-direction of alternating insulating and conducting layers (8), which are in the form of a super-lattice (7).

FIG. 4 illustrates various embodiments for the detection of macromolecules. In (a), a constant potential V(t) (9) generates a dipole which attracts the macromolecule (5), resulting in current flowing between the electrodes. In the embodiment illustrated by (b), the conductance R(t) is measured across the electrodes to detect the macromolecule. Embodiment (c) comprises light (11) from a light source, that generates a conductance in the bound macromolecule (photo-conductance), which can be measured across the electrodes. Alternatively, the light source can be set to generate pulsed light with specific temporal characteristics, which leads to generation of hopping conductance in the macromolecule, which in turn can be measured across the elecrodes.

FIG. 5 illustrates an embodiment, wherein specific binding of macromolecules (5) is achieved by forming canisters, e.g. by laser ablation, through at least one layer of the alternating insulating and conducting layers of the superlattice (7), the binding surface being realized on the inside surface of the canisters.

FIG. 6 illustrates one possible chromatographic embodiment, in which microchannels (13) cross multiple, serially connected superlattices with alternating layers (12) of insulating (1) and conducting (2) material. A macromolecular sample enters through an inlet (14), separation of macromolecules according to their dipoles occurs within the device and the sample subsequently exits at the outlet (15) of the device.

FIG. 7 illustrates a second possible chromatographic embodiment, in which substantially parallel microchannels (20) are formed within one unit of alternating conducting and insulating layers (8) of the superlattice (7). In this embodiment, a sample containing macromolecules (5) enters through an inlet (16), goes through the microchannel and exits at the outlet (19). Optionally, electrodes (17) may be placed perpendicular to the alternating layer (8); the purpose of these electrodes is to reduce laminar flow within the microchannel.

### DESCRIPTION OF THE INVENTION

The object of the device according to the present invention is to selectively bind and detect a single molecule or a group of molecules from a mixture of molecules, preferably macromolecules such as in particular proteins, where the specific binding and detection can be readily tuned and altered to fit a selected molecule or group of molecules. The device is based on the characteristics of most molecules that they contain a characteristic multipole moment that incidentally may be approximated by a set of distributed dipoles. If the molecule or molecules of interest do not contain a characteristic dipole moment, such a dipole moment may be induced by applying a suitable electrical field. Thus, the device can be adapted so that it may be used in the analysis of virtually any molecular species.

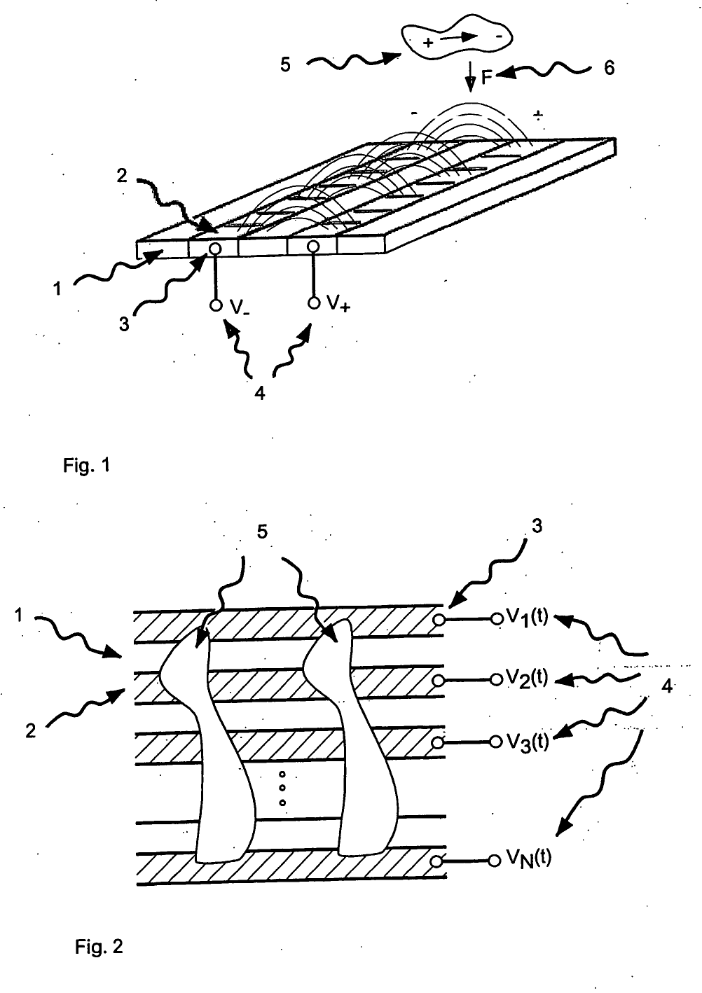

The device is constructed by a set of spatially distributed electrodes, see FIG. 1. The electrochemical potential of the electrodes can be tuned individually or in groups, see FIGS. 1 and 2. The potential of the electrodes may also be time-dependent, so as to optimize binding to macromolecules of interest. The added time-dimension also confers an increased resolution of the device.

The electrodes can be tuned such that they collectively do not result in any charge accumulation. Thus, the resulting field can be described with a set of spatially distributed dipoles, i.e. the electrodes can be viewed as a spatially fixed set of tunable dipoles. The strength and polarization of the dipoles is tunable in both time and space as illustrated in FIG. 2.

Each particular macromolecule has a unique electrostatic profile. The electrostatic profile can be approximated with a set of spatially distributed electrostatic dipoles. The dipoles of the macromolecule interact with the trap by weak short-range Van der Waal forces or dipole-dipole interaction.

For a given apatial configuration of the dipoles in the trap there exists a configuration of their strength and polarization such that the binding energy E_{b} to any given macromolecule is maximized.

At a specific temperature T measured in degrees of Kelvin (K) the macromolecule will be trapped if the maximal binding energy is sufficiently larger than the thermal energy E_{T}=k_{B}T, where k_{B}=1.381×10⁻²³ K·J⁻¹ is the Boltzmann constant The temperature T of the device may be adjusted, both locally, i.e. within a certain area of the device, or globally, i.e. affecting the entire device. This adds an additional variable which may be used to optimize the conditions for any particular embodiment of the device.

Spatial configurations of electrodes that are such that only one or a few electrodes are contained within the area occupied by a typical macromolecule will possibly at the dipole configuration of maximal binding for a specific macromolecule, the target macromolecule, trap a large group of different macromolecules. This binding is not desirable and is due to non-specific binding of macromolecules to the trap. However, as the density of electrodes in the configuration is increased the degree of non-specific binding is expected to decrease until only the target macromolecule becomes trapped. Thus, the device may be configured in a manner suitable for any particular macromolecule or a group of macromolecules.

In any sample containing macromolecules, such as proteins in a blood plasma sample, there is a finite number of distinct molecules. Then a finite number of electrodes will be able to selectively trap any of the molecules in the sample. In a preferred embodiment the number of electrodes used is minimized with the constraint of keeping the spatial configuration simple. In this way the objective with the trap is preferentially obtained.

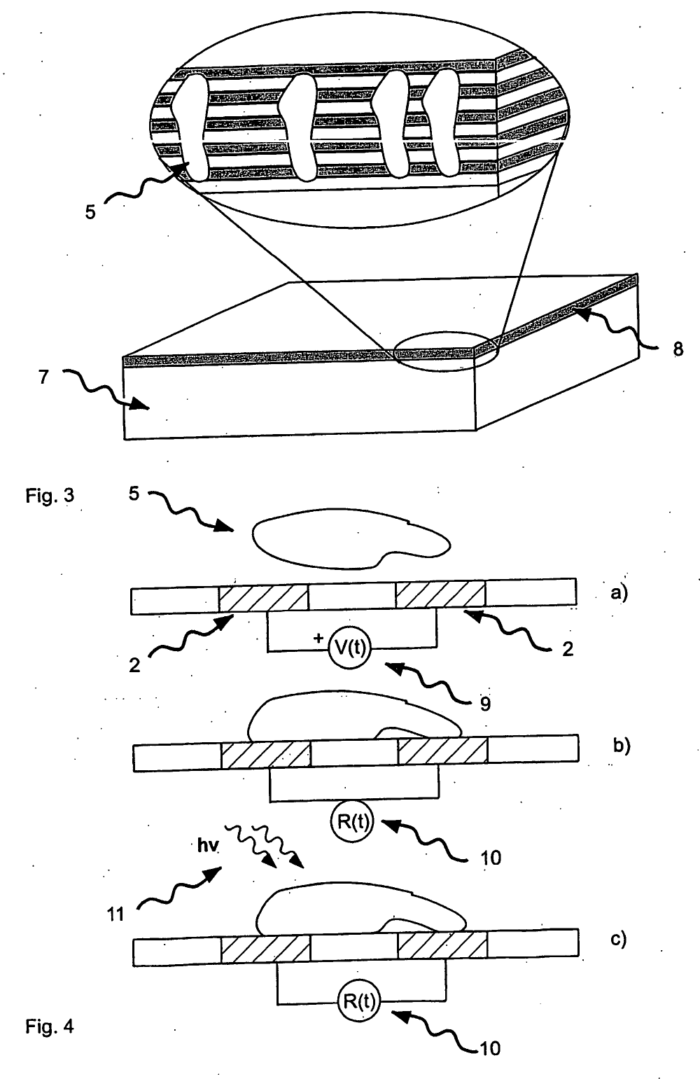

Repeating the pattern of the spatial configuration and linking the appropriate electrodes allows for trapping of many identical macromolecules simultaneously. This is most readily accomplished by doing so periodically in space in one or more dimensions, see FIGS. 2 and 3. In particular, by extending the electrodes in one direction, rails are formed onto which the proteins can bind. Such rails would typically be in the range of about 0.1-10 cm in length, such as on the order of about 1 cm and thereby be able to trap a large number of macromolecules simultaneously, since for example proteins typically range in sizes from about 50 nm to a few hundred nanometers in diameter. The insulating and conducting layers will typically have a thickness in the range of about 0.1 nm to about 1 μm. The particular dimensions of any given embodiment will be highly dependent on the specific configuration of the device.

When a macromolecule is trapped in the single macromolecular trap, FIG. 1, it will be able to short-circuit the electrodes. Keeping the potentials constant a current will flow between the electrodes. The presence of the current indicates that a macromolecule is present in the trap. Any given electrode within the trap may configured so as to serve as a detection electrode. Therefore, the trap is adjustable so that optimal configuration for any given sample may be obtained.

If the macromolecule, in particular a protein, has poor conductance, irradiating the molecule with light at a specific frequency that opens up conductance channels can enhance the conductance. This is termed photo-conductance. The photo-conductance roughly reflects the density of states as a function of energy in the molecule.

The photo-conductance as a function of frequency of the light provides a fingerprint of the macromolecule in the trap. The fingerprint can be used to determine the identity of the different macromolecules present in the trap in cases where the trap-configuration is such that several different macromolecules adhere to the trap.

Consider the multiple trap configuration, FIGS. 2 and 3. Macromolecules that are trapped in this configuration short the electrodes and a current will run through the molecules as the constant potentials are maintained. The current running between the electrodes is proportional to the number of molecules that adhere to the trap. The current can therefore be used as a quantitative measure of the macromolecular concentration in the sample being measured.

The photo-conductance as a function of frequency contains information about the macromolecules attached to the trap. If several kinds of macromolecules adhere simultaneously to the trap, the photo-conductance is used to determine which macromolecules and in what amount they are stuck in the trap. This is achieved by fitting the overall photo-conductance with a linear combination of the photo-conductance of the individual macromolecular species expected to be present in the sample. This requires that the photo-conductance of the individual macromolecules be known for the specific trap configuration.

Thin film sputtering techniques enable production of the trap in a continuous configuration. Modern sputtering techniques allow for controlled growth of conducting element layers or insulating layers with monolayer resolution. The materials used in the conducting and insulating layers may be any organic or inorganic material which has the characteristics required of the layers for any given embodiment of the trap. Thus the conducting layer will typically be comprised of a metal or a semi-conductor, and may in particular be comprised of metals such as silver, platinum, gold, aluminum, copper, molybdenum, platinum, iron, or nickel, while the insulating layer will typically be comprised of an insulator or non-doped semiconductor, and may in particular be comprised of compounds such as magnesium oxide, silica oxide, aluminum oxide, carbides or nitrites. Typical thickness of the monolayers will be in the 0.10-0.20 nm range.

The trap is realized by growing a super-lattice of alternating layers of conductor and insulator materials. The conducting layers should preferably be as thin as possible. In practice a thickness in the range of about 1-10 monolayers, preferably 2-3 monolayers, is ideal. The crystal constant of the insulator and the conductor will typically not match. The substrate, which is composed of the insulating component, carries the governing crystal constant. By sputtering only very thin layers of the conductor, its crystal constant will adapt to the underlying substrate. The insulating layers of the super lattice have to be sufficiently thick such that chosen potential differences between conducting layers can be readily maintained without large power consumption. Tunneling currents can be significant through a few monolayers of insulator, but problems from those are manageable from a thickness in the range of about 3-5 monolayers. Thus the period of the super lattice can in principle be in the range of about 4-15 monolayers, but preferably can start at 5-8 monolayers. Electrodes or conducting layers designed to be a part of the conductance through proteins or other macromolecules may need to be further separated from adjacent conducting layers.

When the super lattice has been grown it is cleaved or polished down such that a surface perpendicular to the growth direction of the super lattice is revealed. On that surface the conducting layers form a pattern of parallel lines or rails. The rails are the electrodes that form the trap in the continuous configuration described above, as shown schematically in FIG. 3.

Electric contact to the conducting layers is readily obtained by sputtering through a series of appropriate masks.

### DETAILED DESCRIPTION OF PREFERRED EMBODIMENTS

The present invention is based on the fact that modern thin film growth techniques on substrates are capable of controlled growth with monolayer resolution. Techniques such as molecular beam epitaxy (MBE) and other sputtering techniques have this capability. Monolayers typically have a thickness of 0.10-0.20 nm. Applying masks that shield the area not to be grown allows for control of the shape of the area grown. The preferred embodiment of the device can be realized applying the aforementioned techniques. The device is constructed by growing alternate layers of electrically insulating and conducting materials. The chosen materials should preferably have similar crystal constants. Otherwise it is difficult to obtain molecular resolution at the interface between the materials. Materials used should also be inert with respect to oxidation, but can in principle be any organic or inorganic material having the necessary properties of the conducting and insulating layers. The device contains at least 2 layers of conducting material separated by insulating layers such that each is connected to a tunable electromagnetic force (EMF). The contacts to the conducting layers of the device are obtained by growing the structure through a set of appropriate masks. The EMF can be tuned by using multi channel commercial digital analogue converters (DACs) that can be controlled with a microcomputer. The EMF may be time-dependent, which lends an additional dimension to the possible configurations of the trap, so as to make it as flexible as possible.

In one embodiment of the device the grown structure is cleaved such that a surface perpendicular to the growth direction is produced, revealing the alternately grown layers. Said surface embodies the active surface of the device that interacts with the macromolecules. The active surface consists of narrow, approximately 0.1-10 nm thick essentially parallel electrodes separated by insulating layers, each of which is in the range of about 0.1-10 nm thick. The overall dimensions of the surface can be in the range of about 0.1-5 cm in width, such as 0.5-2 cm, and in the range of about 0.01-5 cm in height, such as 0.1-1 cm.

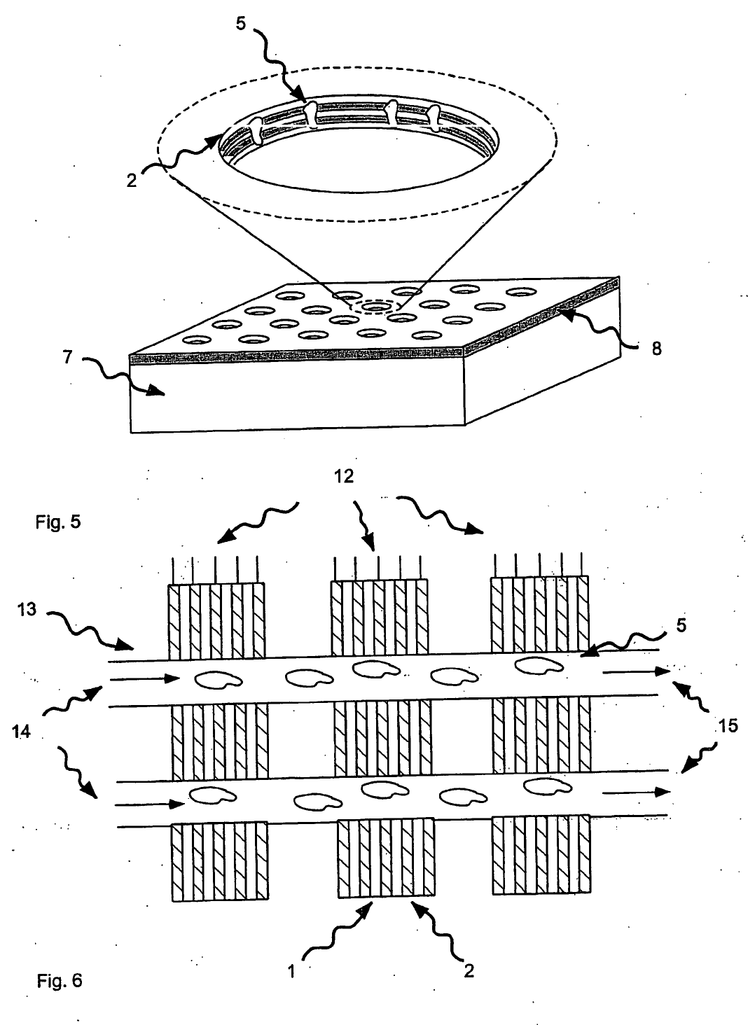

In another embodiment, the device comprises a dense array of canisters which may be formed by e.g. laser ablation, have a width in the range of about 0.1 μm-5 mm, such as 0.5 μm-5 μm, or 5 μm-50 μm, or 100 μm-1 mm, and penetrate one or more of the conducting and insulating layers. In this embodiment, the active surface of the device consists of stacks of circular electrodes within the inner surface of each canister, see FIG. 5.

By tuning the EMFs, a wide range of electrostatic profiles across the active surface can be obtained. The EMFs may also optionally be time-dependent. The electrostatic characteristics of many proteins and other macromolecules are known or can be readily calculated based on the sequence and/or structural data for any given macromolecule. Such data is for example available from public databases for a large number of peptides and proteins. Where detailed structural data is not available the electrostatic characteristics may be approximated through theoretical models. Knowing the electrostatic characteristics of a given type of macromolecule one can theoretically calculate the affinity of the specific macromolecule to the binding site of the device for a given configuration of the EMFs. In this context, consider a sample containing a set of known proteins. Then one can optimize the configuration of the device such that the affinity is maximized for one type of protein while the affinity for the remaining types of proteins in the sample is low in the sense that the affinity to the active site of any given protein molecule is less than k_{B}T.

One aspect of the invention is a method to isolate molecules from samples. After incubating the sample in contact with the device, the device is cleansed using an appropriate medium, such as a washing solution if the sample is liquid, or an inert gas if the sample is gaseous, leaving only the molecules of interest bound to the active surface. The isolated molecules are subsequently released into a receiving medium by turning off the EMFs, or by adjusting the EMF such that a dipole moment is generated that repels the isolated molecules. This may be done repeatedly in order to collect the molecular species of interest from the sample.

Another aspect of the invention provides a method to determine the concentration of a specific molecule in a sample. The molecules that adhere to the active surface at any given time electrically short the spatially separated detector electrodes. Thus currents are necessary in order to maintain the chosen electrostatic configuration. The current necessary to maintain the configuration is proportional to the number of molecules that adhere to the active surface. Using standard samples with known concentrations of the molecule, standard curves are obtained that describe the rate at which the current necessary to maintain the configuration changes as a function of the concentration of molecule in the sample. These curves are subsequently used to determine the concentration of the molecule in unknown samples.

As mentioned earlier, the conductivity of a bound macromolecule is enhanced by irradiating the sample with light either at a fixed frequency or a broad spectrum but at a fixed intensity and duration. The light excites local and extended electron states within the macromolecule leaving open channels that enhance the conduction between the spatially separated detector electrodes through the macromolecule.

The photo-conductance through a specific type of macromolecule as a function of frequency of the light is unique can be utilized, as illustrated in FIG. 4c. The said information essentially reflects the density of electronic states within the macromolecule that can be considered to represent a fingerprint of the macromolecule. By mapping the photo-conductance curves for macromolecules in a sample with samples of known composition, information is obtained that allows for determination of the different types of molecules attached to the active surface of the device. With this information photo-conductance curves reveal which types of macromolecules adhere simultaneously to the device as well as their relative concentration. Quantitative information about the concentration is obtained as well by comparison to photo-conductance curves obtained for samples of known concentration.

The invention can also provide the detection of molecules that have poor or poorly distinct photoconductance curves. This would typically be agents with few extended electronic states; consequently the states are local and tightly bound. In such cases the presence of the molecule adhering to the device is detected by mapping the hopping conductance through the molecule. The hopping conductance is induced by applying pulsed light at a unique frequency or otherwise to the active site of the device and thereby the macromolecules adhering to the device. The concentration of the bound molecules is assessed by comparing the hopping conductance to standard curves obtained by measuring the hopping conductance in samples with known concentrations of a given macromolecule.

As mentioned above, the device can be configured to function as a catalytic site for chemical reactions between at least two macromolecules, e.g. compounds A and B. In this configuration, one group of electrodes on the surface is tuned so as to optimize the affinity to compound A, while an adjacent group of electrodes is tuned such as to optimize the affinity to compound B. The device will thus increase the probability of chemical reactions occurring when placed in samples containing compounds A and B, by bringing the compounds into spatial proximity with each other, and thus increasing their effective concentration. The chemical reaction occurs with increased probability when the compounds are released from the device into close proximity in the surrounding medium.

In another aspect of the invention the device is used as a chromatographic matrix. In this embodiment, a microchannel is placed along the active surface of the device or in such a manner that the channel repeatedly crosses the active surface of the device. The surface can then be tuned in a general manner such that the active surface will have general affinity to macromolecules. For example, one could choose a simple strong dipole moment that is repeatedly turned on and off, so as to bind the macromolecules in a non-specific but reversible manner to the device. The time frequency of the applied dipole will depend on various parameters, such as the drag or viscosity of the medium, the electrical field applied, the specific molecules of interest being analyzed, as well as the size parameters of the microchannel, and temperature. Separation of different macromolecules occurs in the device according to dipole moment as the sample is pumped or by other means passes through the micro channel since the delay will be proportional to the dipole moment of the molecule, as well as inversely proportional to the drag that the medium exerts on the molecule, In an embodiment where the sample is pumped through the microchannel, any mechanical pump capable of pumping the sample in an effective manner through the microchannel can be used. The concentration is detected along the microchannel and distal to the active surface, after the sample has passed through the device, by any conventional detection techniques, such as UV absorption, fluorescence, chemical potential, radioactivity, mass spectrometry, circular dichroism, or any other technique used to detect organic and/or inorganic molecules.

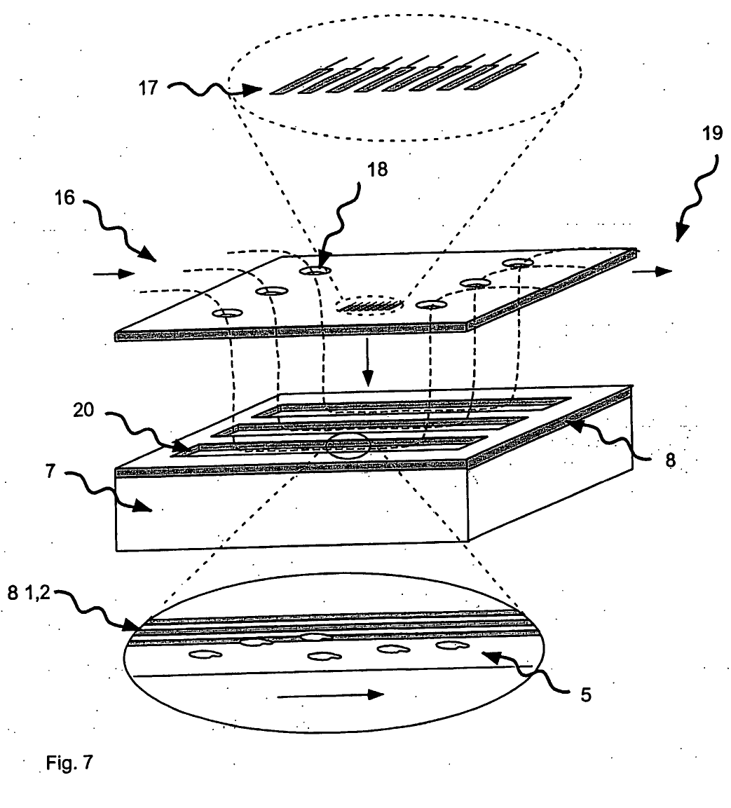

FIGS. 6 and 7 illustrate two possible embodiments of such chromatographic embodiment. In the embodiment shown on FIG. 6, a set of one or several, substantially parallel microchannels 13 cross a plurality of serially connected superlattices 12, each containing alternating layers of insulating 1 and conducting 2 material. The sample enters through an inlet 13 into the device, wherein separation occurs according to the specific dipoles of the molecules in the sample, as they pass through the electromagnetic field generated by the electrodes. Means of detecting said molecules may be set up as or after the sample passes through the outlet 15.

An alternative embodiment showing the device of the invention being used for chromatography is illustrated by FIG. 7. In this embodiment, the sample enters through the inlet 16 and is fed into individual microchannels through a series of openings 18. The individual molecules 5 in the sample are retained according to their dipole moments as the pass through the microchannel 20, which is lined by the electrical rails formed by the alternating layers of insulating and conducting material 8. The microchannels 20 can be configured in any manner suitable for the particular embodiment of the device. Thus, the microchannels may be straight and substantially parallely oriented, as illustrated by the embodiment of FIG. 7. Alternatively, the microchannels may be shaped in any way suitable for optimising flow through the microchannel and effective separation of the molecules in the sample, such as in shape that once or repeatedly crosses the alternating layers of insulating and conducting materials 8.

The sample used in the aforementioned embodiments, or in any other embodiment of a device of the invention, may be liquid, gaseous, or semi-liquid in nature, such as a gel matrix, hydrogel or any other chemical carrier suitable for molecular analysis according to the invention. Each embodiment will be optimized to suit the particular use. Although the device is ideally suited for the analysis of macromolecules, any molecular species fulfilling the requirements of binding to the active surface and which can be detected by the aforementioned techniques can be analyzed by the invention. Gaseous samples, in particular, may be analyzed using the device in a configuration for receiving such samples. Thus, the device of the invention can be configured to act as an ‘electrical nose’, in which qualitative and quantitative analysis of the molecular composition of gaseous samples may be performed in a manner analogous to that illustrated by the embodiments described above. Thus the device can be configured to suite the purpose of a range of analyses, and the examples shown above should only be seen as examples of possible embodiments of the invention, and, as such, not limiting for the range of applications possible within the scope of the invention.

1. A device with tunable affinity for molecules, comprising:
a. a substrate with a surface;
b. a plurality of locally substantially parallel electrodes along said surface, such as about 0.3 nm to about 5 nm, each of said electrodes being connected to a tunable EMF source, such that a specific electrostatic environment perpendicular to said electrodes is created, extending along the electrodes, and providing a continuous binding area for molecules in contact with the binding area;
wherein by tuning the independently tunable EMF sources a specific affinity or repulsion is obtainable for molecules with specific electrostatic properties.

2. The device of claim 1, wherein adjacent electrodes are separated by a distance in the range of about 0.1 to about 10 nm, such as about 0.2 to about 8 nm, such as about 0.3 to about 5 nm, including about 0.5 to about 3 nm.

3. The device of claim 1, wherein the surface is formed perpendicular along the growth direction of alternately frown layers of insulating and conducting material.

4. The device of claim 1, wherein said surface is formed by an array of canisters penetrating at least one layer of alternating insulating and conducting material, in which the inner surface of said canisters forms said surface.

5. The device of claim 1, wherein the molecules are macromolecules.

6. The device of claim 5, wherein the macromolecules are selected from the Group consisting of polypeptides, proteins and nucleic acids including DNA and RNA.

7. A method of isolating molecules based on their electrostatic properties, the method comprising:
a. providing a substrate having a plurality of locally substantially parallel electrodes, each electrode being independently connected to a tunable EMF source;
b. tuning said electrodes to individually selected potentials, thereby creating a specific electrostatic profile across the electrodes to generate a molecule-specific surface;
c. contacting a medium comprising molecules with the substrate to allow binding of specific molecules having an affinity to said surface;
d. separating the medium from the substrate so as to separate the molecules bound to the substrate from non-binding molecules in the medium;
e. isolating said specific molecules by adjusting said EMF source to release by repulsion or by other means said molecules into a receiving medium brought in contact with the substrate.

8. The method of claim 7, further comprising the step of repeatedly releasing specifically bound molecules from said substrate into a receiving medium and subsequently bringing said substrate in contact with the medium containing molecules, so as to effectively remove specific molecules from the medium.

9. The method of claim 7, further comprising the step of detecting said molecules by observing the change in current through one or more of said electrodes.

10. The method of claim 9, wherein quantitative assessment for said binding molecules is obtained by comparing the current necessary to maintain the electrostatic configuration of the surface to the corresponding current obtained using samples with known concentration of molecules identical to said binding molecules.

11. The method of claim 7, further comprising the step of detecting said binding molecules by irradiating the molecule with light and measuring the conductivity of said molecules.

12. The method of claim 10, further comprising the step of measuring the conductivity of said binding molecules as a function of frequency of the light, thus generating a photo-conductance curve of said molecules.

13. The method of claim 11, further comprising the step of quantitative determination of said binding molecules, by mapping photo-conductance curves for macromolecular species present in the sample to photo-conductance curves of samples of known concentration containing said macromolecular species.

14. The method of claim 7, further comprising the step of detecting said binding molecules by irradiating the molecules with pulsed light and measure the hopping conductance of said molecules.

15. The method of claim 14, further comprising the step of quantitative determination of said binding molecules, wherein the hopping conductance for the molecules is compared to the hopping conductance of said molecules in a sample of known concentration.

16. A method of catalyzing chemical reactions between molecules comprising:
a. providing a substrate having a plurality of locally substantially parallel electrodes, each electrode independently connected to a tunable EMF source;
b. tuning said electrodes to individually selected potentials, thereby creating a specific electrostatic profile across the electrodes to generate a molecule-specific surface;
c. contacting a medium comprising molecules with the substrate to allow binding of molecules specific for the chemical reaction and having an affinity to said surface;
d. release of said molecules by adjustment of said EMF source into close spacial proximity of said surface;
e. chemical reaction between said molecules accelerated by the increase in local concentration of said molecules.

17. The method of claim 16, further comprising the step of adjusting the EMF in substantially parallel electrodes in an adjacent fashion, so as to generate adjacent specific binding sites for molecules specific for the chemical reaction.

18. A method of separating molecules, comprising:
a. providing a substrate having a plurality of locally substantially parallel electrodes, each electrode being independently connected to a tunable EMF source;
b. tuning said electrodes so as to generate a non-specific electrostatic profile across the electrodes, thus generating a non-specific macromolecular surface;
c. placement of a micro-channel along said surface;
d. allowing a medium comprising molecules to pass through the micro-channel, t hereby allowing separation of molecules according to their dipole moment.

19. The method of claim 18, wherein the tunable EMF is applied in a cyclic manner so as to generate a time-dependent non-specific macromolecular surface, thus allowing reversible non-specific binding to the surface.

20. The method of claim 18, further comprising the step of detecting said molecules by applying a detection technique along said micro-channel distal to said surface, said technique being selected from the group consisting of UV spectrophotometry, circular dichroism, fluorescence spectrophotometry, mass spectrometry, chemical potential, radioacitivity.

21. The method of claim 7, wherein the molecules are macromolecules.

22. The method of claim 21, wherein the macromolecules are selected from the group consisting polypeptides, proteins and nucleic acids including DNA and RNA.

23. (canceled)

24. A method of producing a device with tunable affinity for molecules, comprising:
a. growing a superlattice structure of alternate layers of electrically insulating and conducting materials;
b. cleaving or polishing the superlattice structure to obtain a surface essentially perpendicular to the growth direction of the superlattice structure;
c. connecting two or more of the conducting layers individually to tunable EMF sources, to obtain substantially parallel electrodes extending along said surface, which electrodes are individually or collectively tunable to a desired potential.

25. The method of claim 24, further comprising the step of forming an array of canisters penetrating at least one of said alternate layers of electrically insulating and conducting material.
